(19) United States
(12) Patent Application Publication
(10) Pub. No.: US 2003/0023236 A1
(43) Pub. Date: Jan. 30, 2003

(54) Cooled tip laser catheter for sensing and ablation of cardiac arrhythmias

(75) Inventors: Ashok Gowda, Houston, TX (US); Roger McNichols, Pearland, TX (US); Sohi Rastegar, Falls Church, VA (US)

Correspondence Address: SIMON, GALASSO & FRANTZ PLC, P.O. BOX 26503, AUSTIN, TX, 78755-0503, US

(73) Assignee: Bio Tex

(21) Appl. No.: 10/208,222

(22) Filed: Jul. 30, 2002

Related U.S. Application Data
(60) Provisional application No. 60/308,620, filed on Jul. 30, 2001.

(51) Int. Cl. A61B 18/22

(52) U.S. Cl. 606/15

(57) ABSTRACT

The disclosures made herein relate to methods and equipment adapted for treatment of cardiac arrhythmias and for limiting, if not preventing, damage to surface tissue while coagulating tissue within the myocardium. In one embodiment of the disclosures made herein, a cooled tip laser catheter system includes an energy delivery apparatus, a laser apparatus and a cooling medium supply apparatus. The energy delivery apparatus includes a flexible tubular housing, a tip assembly and an optical waveguide. The flexible tubular housing includes a plurality of lumens therein extending between a proximal end and a distal end of the flexible tubular housing. The tip assembly includes a tip body attached at a first end thereof to the distal end of the flexible tubular housing and an optical window mounted at a second end of the tip body. The circulation chamber is defined within the tip body between the distal end of the flexible tubular housing and the optical window. The optical waveguide is mounted within a first of said lumens, wherein a distal end of the optical waveguide is exposed within the circulation chamber. The laser apparatus is attached to the energy delivery apparatus in a manner enabling laser light to be supplied to and transmitted by the optical waveguide. The cooling medium supply apparatus is attached to the energy delivery apparatus in a manner enabling cooling medium to be circulated through the circulation chamber.

### FIELD OF THE DISCLOSURE

The disclosures herein relate generally to equipment (e.g., systems, apparatuses and devices) and methods for treating cardiac arrhythmias, and more particularly to treatment of cardiac arrhythmias using methods and equipment adapted for limiting damage to organ surface tissue while coagulating myocardium tissue.

### BACKGROUND

The American Heart Association estimates that approximately 1.5 million individuals suffer myocardial infarctions annually in the United States, of which, approximately 1 million survive. Myocardial infarctions generally result in cardiac arrhythmias, including Ventricular Tachycardia (VT), and are responsible for 400,000 cases of sudden death in the U.S. each year. Approximately one third of the survivors of a myocardial infarction are at risk of suffering an episode of VT within the following year after such a myocardial infarction.

A normal heart contraction is the coordinated result of organized electrical signals generated by the heart's natural pacemaker, the sino-atrial node (SA node), and conducted throughout remaining tissue of the heart. The SA node initiates an electrical signal that causes atria (i.e., upper chambers) of the heart to contract, providing a primer volume that aids in filling ventricles (i.e. lower chambers) of the heart. The electrical signal continues to the atrioventricular node (AV node). The AV node serves as a delay for the electrical signal, allowing the ventricles to fill with blood, and then facilitates the organized spread of the electrical signal to the ventricles, causing them to contract. Ventricular contraction distributes deoxygenated blood to the lungs from the right ventricle and oxygenated blood to the rest of the body from the left ventricle.

VT is a life-threatening condition characterized by abnormally high rate of ventricular contraction. Most cases of VT are the result of myocardial infarctions caused by coronary artery disease. The abnormally high rate of ventricular contraction associated with VT prevents the ventricles from filling with sufficient amounts of blood prior to each contraction, resulting in insufficient blood flow to heart muscles. Such an insufficient blood flow often results in a portion of the muscle (usually in the left ventricle) dying and forming scar tissue. The border of a myocardial infarction generally comprises an irregular mix of healthy cells and scar tissue. Abnormal signals responsible for initiation of VT generally originate at this border of the myocardial infarction.

Many current therapies for VT are not curative and often have undesirable side effects. Anti-arrhythmic drugs are currently the most common form of treatment for VT. In 1989, a multi-center randomized trial evaluating anti-arrhythmic capabilities of several drugs indicated that many of the drugs actually induced the occurrence of VT. Additionally, toxic side effects including pulmonary fibrosis, corneal micro deposits and liver dysfunction prevent long-term usage of anti-arrhythmic drugs.

An automatic Implantable Cardioverter Defibrillator (ICD) has also become a standard therapy in treatment of VT. The ICD detects and stops an arrhythmia by applying high-energy defibrillation pulses to the heart to reset the heart's normal rhythm. While the automatic ICD is an effective means for stopping the arrhythmia, it does not directly address tissue responsible for the arrhythmia, and therefore is not curative. Side effects from the use of an automatic ICD include pain associated with high-energy defibrillation pulses, discomfort associated with the implant, risk of infection and a risk associated with outside interference from electronic devices. Furthermore, many patients with an automatic ICD must remain on anti-arrhythmic drugs in an attempt to minimize the number of episodes of VT.

Presently, the only curative treatment for VT is removal or destruction of the tissue area responsible for initiating the arrhythmia. Catheter ablation has become standard treatment for many types of arrhythmia. In order to successfully perform catheter ablation, electrical mapping techniques must first be used to locate the arrhythmogenic area (i.e., focus) or areas (i.e., foci) of tissue responsible for generating the arrhythmia. Once an area or areas of tissue responsible for generating the arrhythmia is identified, catheter ablation is used to irreversibly damage or destroy such area or areas by applying energy (e.g., via laser, RF, microwave, etc) to the myocardium, resulting in thermal heating and the creation of a permanent lesion.

Catheter ablation using radio-frequency energy has become the treatment of choice for supraventricular tachycardias, in which the site responsible for generating the ventricular arrhythmia originates from a site above the ventricles. These sites are generally located in areas close to the interior surface of the heart where the radio-frequency energy is applied and therefore do not require significant lesion depth for effective treatment. Catheter-based ablation is a potentially curative technique for patients with ventricular tachycardia. However, to date, catheter-based ablation for ventricular tachycardia has not had the same high success rates seen in patients with other types of arrhythmia. One reason for such limited success is that critical areas of the electrical circuit responsible for VT may traverse tissue in the midmyocardium or subepicardial region of the heart that are relatively deep with respect to the endocardial surface where energy from the catheter is normally applied.

A number of energy sources including, direct current (DC), radio-frequency (RF), ultrasound, microwave, and laser have been investigated for use in coagulation of myocardial tissue responsible for generating VT. The energy source most commonly used for catheter ablation of arrhthmogenic foci is RF energy with frequency between about 300 kHz and about 1 MHz. This frequency range avoids depolarization of myocytes and ensures resistive heating. RF energy is normally delivered between a tip electrode at the distal end of a catheter and a dispersive electrode located on the patient's body. This approach to delivering RF energy provides maximum dissipation of energy and results in resistive heat formation at the tip electrode in contact with the endocardium.

The magnitude of direct resistive heating (e.g., via a source such as DC, RF, ultrasound and microwave) is restricted to a narrow region of tissue within 2-3 mm of the electrode. Therefore, the tip electrode essentially acts as a local heat source with the majority of lesion formation being due to heat conduction from the superficial tissue layers. Additionally, surface heating with RF develops rapidly, which can lead to boiling of blood in contact with the tip electrode and coagulum formation on the electrode tip surface, causing a sudden rises in impedance. Such a sudden rise in impedance can result in phenomena such as electrical arcing, charring and catheter adherence to the myocardium. These phenomena cause embolic and thrombotic events, reduce current flow and hence deeper tissue heating, and frequently mandate removal of the catheter during the procedure in order to clean its tip.

Attempts to overcome such limitations associated with direct resistive heating have been made by providing a method of cooling the tip electrode in contact with the myocardium during energy delivery. While such a cooling approach improves the safety of radio frequency-based procedures, it is designed primarily to prevent overheating of the tip electrode and to allow longer deposition of energy. However, the conductive and convective properties within the myocardium limit lesion depth due to the short penetration depth of the RF energy. Accordingly, the resulting lesion will still have most of its volume concentrated near the surface adjacent to where the energy is being applied.

Laser induced photocoagulation has also been investigated as a method of creating large myocardial lesions. Properties of laser induced coagulation such as the ability to transmit light through fiber optics, and the deeper penetration of photons into myocardial tissue make laser photocoagulation an attractive means for ablation of tissue causing VT. Additionally, myocardial lesions created with laser energy appear ideally suited for ablation of tissue causing VT as they are large and discrete. A large and discrete myocardial lesion enhances the likelihood of successful ablation by effectively and reliably decoupling healthy tissue from scar tissue, thus precluding or sufficiently minimizing abnormal signals responsible for initiation of VT that generally originate at the border defined by healthy tissue and scar tissue in a myocardial infarction.

Various investigators have conducted a comparison of laser and RF catheter ablation of ventricular myocardium in non-human subject. The results of the comparison indicated significant side effects of using RF, including intramural bleeding, tissue rupture, dissociation of myocardial fibers, and tissue vaporization with crater and thrombus formation. Trans-catheter application of laser light at 1064 nm, however, produced significantly larger and more reproducible lesions than with RF current, and had fewer undesirable effects on the ventricular wall.

To date, conventional laser-based approaches have failed to gain clinical acceptance as the treatment of choice for patients with VT. One draw back to conventional laser approaches is the requirement for the use of small core diameter fiber optics. Small core diameter fiber optics beneficially allows sufficient flexibility for navigation of the distal end of the catheter into and around the ventricles from a percutaneous approach.

However, when used in contact with the tissue surface, a small core fiber results in a high power density during application of the laser energy and therefore tends to result in charring and vaporization of the underlying tissue. These physical changes in tissues create a number of problems. First, charring limits heat deposition within the tissue volume by absorbing incident light energy, limiting the extent of coagulation. As charred tissue continues to absorb light, its temperature continues to rise and further coagulation of deeper layers is strictly dependent on heat conduction away from the charred tissue. Although significantly large and deep lesions can be created in this fashion, the morphology of the resulting lesion is sometimes undesirable. Second, tissue char and vaporization of the endocardial surface can give way to embolic and thrombotic events that pose severe risk to the patient. Finally, the associated high temperatures can also result in melting of catheter tips and fiber optics with degradation of optical performance and significant ensuing risks for patients.

Interstitial coagulation of myocardium has been investigated using interstitial laser and RF energy sources. Although effective in the creation of deep lesions, these methods require precise penetration of the delivery source into the myocardial tissue and therefore run the risk of perforations resulting in life threatening complications such as cardiac tamponade. Furthermore, mechanical damage to the endocardium resulting from fiber penetrations may provide a stimulus for thrombus formation.

Methods to prevent char and enhance lesion size during laser irradiation of tissue have been reported. Various investigators have conducted studies using chilled water to cool the surface of tissue while irradiating it with the Nd:YAG laser. These studies have demonstrated that by cooling the surface during laser irradiation, the zone of thermal damage can reach deeper tissue layers while preserving the superficial layers. At least a portion of the unwanted effects described above can be eliminated or at least mitigated sufficiently by removing heat from the irradiated tissue and maintaining laser delivery below thresholds that might cause intense vaporization of sub-surface tissue.

Based on the forgoing discussion, it will be appreciated that equipment and/or a method adapted for creating myocardial lesions for curative treatment of VT in a manner that overcomes limitations associated with conventional VT treatment approaches are useful and advantageous.

### SUMMARY OF THE DISCLOSURE

The disclosures made herein relate to methods and equipment adapted for treatment of cardiac arrhythmias and for limiting, if not preventing, damage to surface tissue while coagulating tissue within the myocardium. In one embodiment of the disclosures made herein, a cooled tip laser catheter system includes an energy delivery apparatus, a laser apparatus and a cooling medium supply apparatus. The energy delivery apparatus includes a flexible tubular housing, a tip assembly and an optical waveguide. The flexible tubular housing includes a plurality of lumens therein extending between a proximal end and a distal end of the flexible tubular housing. The tip assembly includes a tip body attached at a first end thereof to the distal end of the flexible tubular housing and an optical window mounted at a second end of the tip body. The circulation chamber is defined within the tip body between the distal end of the flexible tubular housing and the optical window. The optical waveguide is mounted within a first of said lumens, wherein a distal end of the optical waveguide is exposed within the circulation chamber. The laser apparatus is attached to the energy delivery apparatus in a manner enabling laser light to be supplied to and transmitted by the optical waveguide. The cooling medium supply apparatus is attached to the energy delivery apparatus in a manner enabling cooling medium to be circulated through the circulation chamber.

An object of equipment and methods in accordance with at least one embodiment of the disclosures made herein is to facilitate creation of large and deep lesions in myocardial tissue, resulting in destruction of arrhythmogenic foci.

Another object of equipment and methods in accordance with at least one embodiment of the disclosures made herein is to provide improved facilitation of thermal treatment of myocardial tissue in a manner that limits thermal and mechanical damage to the endocardium while creating a controlled lesion in tissue underlying the endocardium.

Another object of equipment and methods in accordance with at least one embodiment of the disclosures made herein is to provide a means for delivering laser energy that includes cooling features optimized to allow transport of laser light to the target tissue while removing heat from and preserving tissue upon which the laser light is initially incident.

Another object of equipment and methods in accordance with at least one embodiment of the disclosures made herein is to utilize a cooling medium for preventing overheating of an optical waveguide and tissue in direct contact with an optical window through which laser light is delivered via the optical waveguide to the tissue.

Another object of equipment and methods in accordance with at least one embodiment of the disclosures made herein is to facilitate simultaneous monitoring of electrical activity in tissue during application of laser light.

Another object of equipment and methods in accordance with at least one embodiment of the disclosures made herein is to provide a means for reducing power density of laser light on incident tissue while maintaining flexibility and maneuverability of a catheter component through which the laser light is delivered.

Another object of equipment and methods in accordance with at least one embodiment of the disclosures made herein is to facilitate maximal energy deposition combined with controlled cooling medium temperature and/or flow for enabling maximum lesion size to be achieved.

Another object of equipment and methods in accordance with at least one embodiment of the disclosures made herein is to facilitate correlation of electrical signals of at least one feedback variable to levels of laser-induced damage in tissue during therapy.

Another object of equipment and methods in accordance with at least one embodiment of the disclosures made herein is to provide a light delivery component (e.g., an optical fiber) that is removeably attached to a flexible tubular housing of an energy delivery apparatus.

Another object of equipment and methods in accordance with at least one embodiment of the disclosures made herein is to provide an energy delivery apparatus adapted for limiting, if not preventing, forward translation of a laser light delivery component within a tubular housing of the energy delivery apparatus.

Another object of equipment and methods in accordance with at least one embodiment of the disclosures made herein is to provide an energy delivery apparatus adapted for enabling controllable translation of a laser light delivery component within a tubular housing of the energy delivery apparatus.

Another object of equipment and methods in accordance with at least one embodiment of the disclosures made herein is to provide an energy delivery apparatus adapted for providing an essentially constant spot size on an optical window of the energy delivery apparatus during deflection of a catheter component of the energy delivery apparatus.

Another object of equipment and methods in accordance with at least one embodiment of the disclosures made herein is to provide an energy delivery apparatus adapted for enabling a variable spot size on an optical window of the energy delivery apparatus.

Another object of equipment and methods in accordance with at least one embodiment of the disclosures made herein is to provide an energy delivery apparatus adapted for facilitating electro-physiological mapping of a heart, wherein such facilitation may include applying an electrical potential for carrying-out a heart pacing protocol and/or facilitating measuring of an electrical signal generated by the heart.

Another object of equipment and methods in accordance with at least one embodiment of the disclosures made herein is to provide an energy delivery apparatus wherein respective faces of an optical window and a tip body of the energy delivery apparatus are essentially flush, thereby enhancing thermal and electrical contact area.

Another object of equipment and methods in accordance with at least one embodiment of the disclosures made herein is to enable a VT treatment procedure via a percutaneous approach under a known guidance technique for achieving permanent correction of arrhythmia generating myocardial defects.

These and other objects of equipment and methods in accordance with at least one embodiment of the disclosures made herein will become more readily apparent from the accompanying drawings and from the detailed description that follows.

### BRIEF DESCRIPTION OF THE DRAWING FIGURES

FIG. 1A is a plan view of an energy delivery apparatus in accordance with an embodiment of the disclosures made herein.

FIG. 1B is a partial fragmentary side view depicting an embodiment of a distal portion of the energy delivery apparatus depicted in FIG. 1A.

FIG. 1C is cross sectional view taken along the line 1C-1C in FIG. 1B.

FIG. 1D is a partial fragmentary side view depicting a proximal portion of the energy delivery apparatus depicted in FIG. 1A.

FIG. 2A is a diagrammatic view depicting an embodiment of a method of treatment with an energy delivery apparatus of a Cooled Tip Laser Catheter (CTLC) system in accordance with the disclosures made herein.

FIG. 2B is an enlarged diagrammatic view depicting the effect of surface cooling on lesion size and depth resulting from treatment with an energy delivery apparatus in the method depicted in FIG. 2A.

FIG. 3A is a side view depicting an optical waveguide in accordance with an embodiment of the disclosed herein, wherein the optical waveguide includes a reverse tapered portion at its distal end.

FIG. 3B is a side view depicting an optical waveguide in accordance with an embodiment of the disclosed herein, wherein the optical waveguide includes a ball-ended lens at its distal end.

FIG. 3C is a side view depicting an optical window in accordance with an embodiment of the disclosed herein, wherein the optical window includes contoured surfaces.

FIG. 3D is a side view depicting an optical window in accordance with an embodiment of the disclosed herein, wherein the optical window includes a surface adapted for diffusing laser light.

FIGS. 3E and 3F are partial fragmentary side views depicting an embodiment of an energy delivery apparatus including a plurality of arms adapted for grasping tissue at a treatment site to aid in maintaining contact between an optical window of the energy delivery apparatus and a treatment site.

FIG. 3G is a partial side view depicting an embodiment of a tip assembly including a plurality protruding members.

FIG. 4 is a block diagram view depicting an embodiment of a Cooled Tip Laser Catheter (CTLC) system in accordance with the disclosures made herein.

FIG. 5 is a diagrammatic view depicting an embodiment of a geometric representation of a thermal delivery model in accordance with the disclosures made herein.

### DETAILED DESCRIPTION OF THE DISCLOSURE

The disclosures made herein relate to various aspects of facilitating treatment of Ventricular Tachycardia (VT) in a manner that overcomes limitations associated with conventional methods and equipment for such treatment. Methods and equipment in accordance with embodiments of the disclosures made herein result in the creation of deep lesions without thermal or mechanical damage to the endocardium or coagulation of surrounding blood flow. Lesions created using methods and equipment in accordance with the disclosures made herein are homogeneous without significant charring of tissue as this could limit energy deposition and hence lesion size while at the same time promote dangerous embolic events. Such lesions are also large and deep enough to destroy the aberrant electrical pathway responsible for episodes of VT. Furthermore, such lesions are discrete, thereby limiting irreversible damage in normal cardiac tissue while still minimizing the potential for arrhythmias from originating from the border between healthy tissue and scar tissue.

Various aspects of Cooled Tip Laser Catheter (CTLC) systems are disclosed herein. Such CTLC systems include various apparatuses and devices. Furthermore, CTLC systems in accordance with embodiments of the disclosures made herein facilitate procedures suitable for relatively safe and effective treatment of Ventricular Tachycardia (VT).

CTLC systems as disclosed herein offer numerous advantages over conventional methods and equipment for treating VT. These numerous advantages include, but are not limited to, the following. One advantage is that large subendocardial laser lesions may be created with nominal or no damage (e.g., char, carbonization, or other unwanted tissue disruption) of the endocardial surface. Lesions originate on average at 1 mm below the endocardial surface, thus enhancing the likelihood of reaching deeper foci responsible for the initiation of VT. Surface cooling techniques in accordance with the disclosures made herein offer an effective means for enabling lesion size to be optimized while preventing unwanted thermal effects. Another advantage is that electro-physiologic monitoring may be performed simultaneously during delivery of laser energy. Yet another advantage is that resulting lesions are well circumscribed and discrete, minimizing the potential for pro-arrhythmia events. Still another advantage is that there is no significant increase in the potential for early rhythm disturbances in patients relative to control patient undergoing similar surgical procedures. A further advantage is that a relatively low-power, low-cost and safe laser apparatus is adapted for enabling large therapeutic lesions to be created.

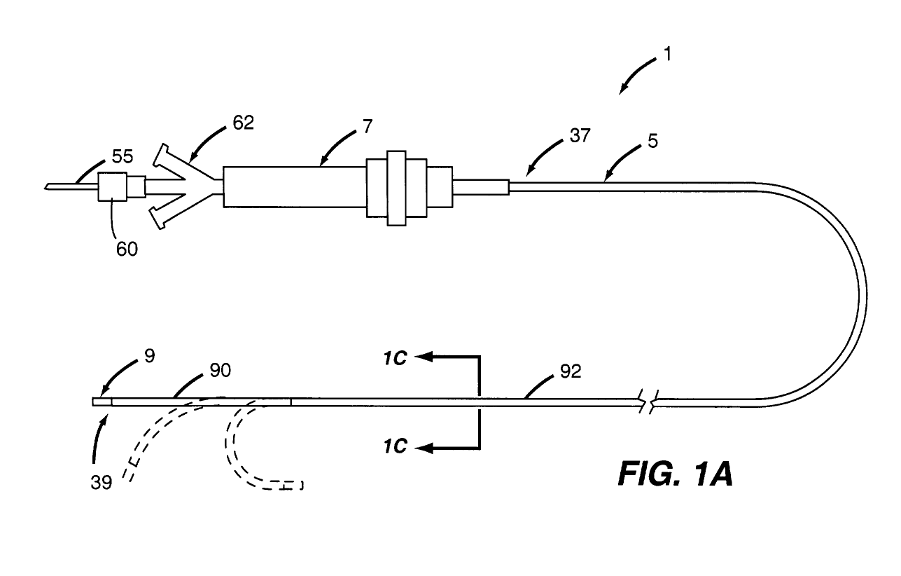

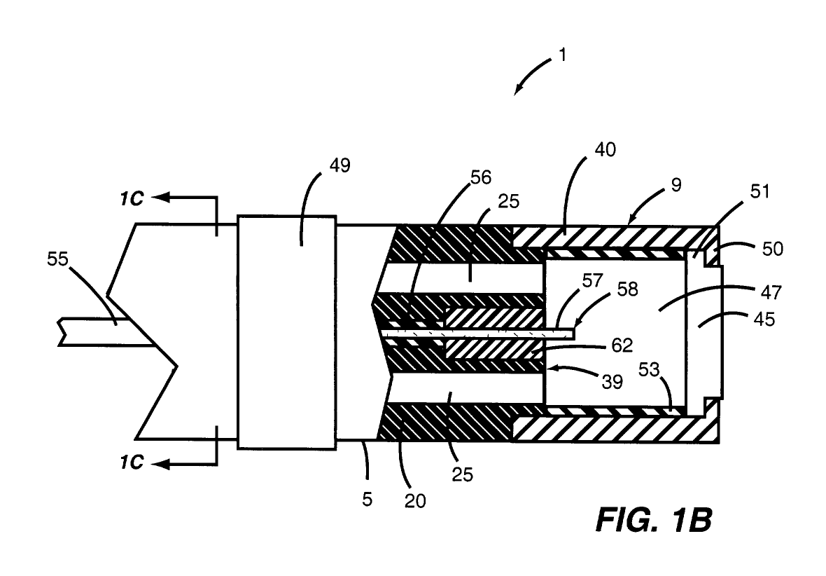

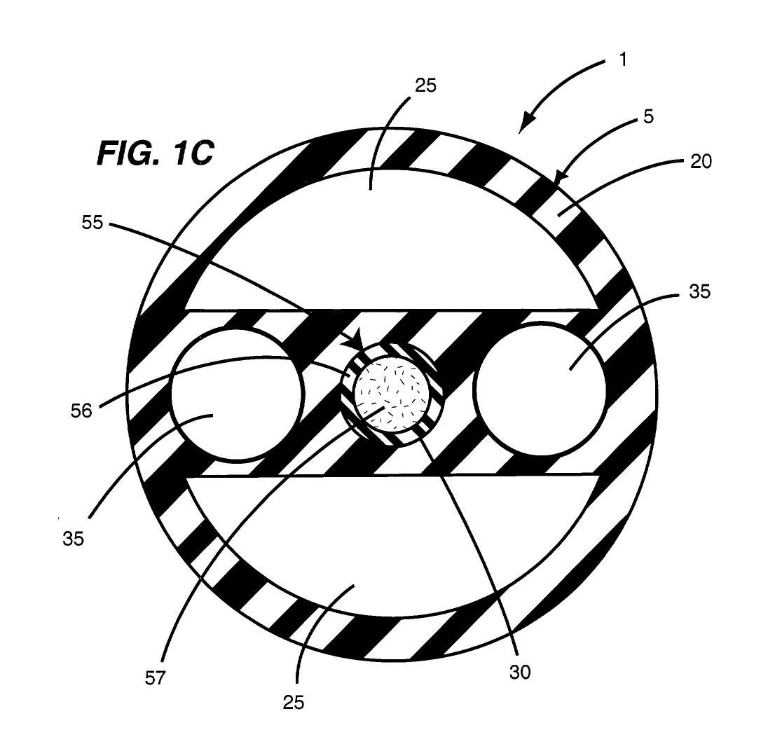

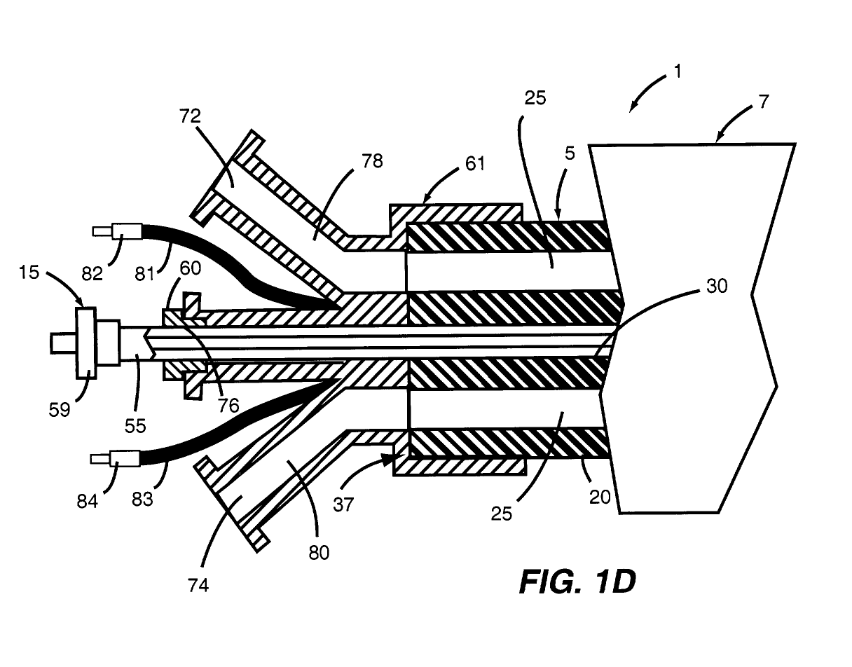

Turning now to the drawing figures, an embodiment of an energy delivery apparatus 1 of a CTLC system in accordance with the disclosures made herein is depicted in FIGS. 1A-1D. The energy delivery apparatus 1 includes a catheter component 5, a handle 7, a tip assembly 9, and laser light delivery component 15. The catheter component 5 is disposed between the handle 7 and the tip assembly 9. The laser light delivery component 15 extends within the catheter component 5.

Referring to FIGS. 1B through 1D, the catheter component 5 includes a flexible tubular housing 20 having a plurality of cooling medium lumens 25, a central lumen 30, and a plurality of auxiliary lumens 35 provided therein. The cooling medium lumens 25, the central lumen 30 and the auxiliary lumens 35 extend between a proximal end 37 and a distal end 39 of the flexible tubular housing 20. The cooling medium lumens 25 and the auxiliary lumens 35 are examples of peripherally located lumens relative to the central lumen 30. The cooling medium lumens 25 facilitate delivery and return of a cooling medium (e.g., water, saline, carbon dioxide, etc) to and from the tip assembly 9. The central lumen 30 facilitates housing of the laser light delivery component 15. The auxiliary lumens 35 facilitate housing of flexing wires, conductors or both. For example, a first one of the auxiliary lumen 35 may facilitate housing of a flexing wire and a second one of the auxiliary lumen may facilitate housing of at least one conductor. Suitable materials for the flexible tubular housing 20 include, but are not limited to, flexible radio-opaque and non-radio-opaque medical grade thermoplastic tubing such as polyurethane, polyethylene, polypropylene, silicone, nylon, PVC, PET, PTFE, ABS, PC PES, PEEK, FEP, and other biocompatible polymers known to those skilled in the art.

The cooling lumens 25 have a truncated semi-circular cross-sectional shape, as depicted in FIG. 1C. Such a truncated semi-circular cross-sectional shape allows for an increased volumetric flow capacity relative to a lumen with a circular cross-sectional shape that is positioned between the central lumen 30 and an exterior surface of the flexible tubular housing 20 (e.g., one of the auxiliary lumens 35). It is contemplated herein that cross-sectional shapes other than a truncated semi-circular cross sectional shape (e.g., an oval, truncated annulus, ellipse, etc.) may be implemented for providing the cooling lumens 25 with increased volumetric flow capacity relative to a circular cross-sectional shape. It is also contemplated herein that in certain applications, the cooling lumens 25 may have a circular cross-sectional shape and support a required volumetric flow capacity.

Referring to FIG. 1B, the tip assembly 9 includes a tip body 40 and an optical window 45. An electrode member, such as a tube made from an electrically conductive material, is an example of the tip body 40. A circulation chamber 47 is defined within the tip body 40 between the distal end 39 of the flexible tubular housing 20 and the optical window 45. The optical window 45 is adapted to be both highly transparent to the wavelength of light emitted from the laser light delivery component 15 and highly conductive to heat. Suitable materials for the optical window include sapphire, synthetic diamond, fused silica, BK7, and other materials known to be suitable for optical windows in laser applications. Preferably, respective faces of the optical window 45 and the tip body 40 are essentially flush, thereby enhancing thermal and electrical contact area.

A proximal sensing member 49 is located on the tubing housing 20 at a respective intermediate location between the proximal end 37 and the distal end 39 of the flexible tubular housing 20. An electrode member, such as a tube made from an electrically conductive material, is an example of the proximal sensing member 49. It is contemplated herein that one or more other proximal sensing bodies may be provided in a similar manner as the tip body 49. The tip body 40 and the proximal sensing member 49 are preferably platinum or stainless steel, but may be made from any suitable electrically conductive material. The tip body 40 and proximal sensing member 49 each have a respective electrical conductor attached thereto for enabling electrical signals to be transmitted between the proximal end 37 and the distal end 39 of the tubing housing 20. It is contemplated herein that an electrically conductive flex wire may be attached to the tip body 40.

The tip body 40 includes a shoulder portion 50 that engages a mating shoulder portion 51 of the optical window 45, thereby providing mechanical support, sealing and a relatively large engagement area between the tip body 40 and the optical window 45. A support ring 53 is positioned between the distal end 39 of the tubing housing 20 and the optical window 45 for providing additional mechanical support for the optical window 45. The support ring 53 may extend partially or fully between the distal end 39 of the tubing housing 20 and the optical window 45. The support ring 53 is an example of an optical window support member.

It is contemplated herein that the tip body 40 may be attached to the flexible tubular housing 20 by means such as crimping, adhesive bonding, thermal bonding, ultrasonic welding, laser welding, shrink fitting, press-fitting and the like. It is also contemplated herein that the optical window 45 may be adhesive bonded to the tip body 40. Examples of suitable, commercially available medical grade epoxy for adhesive bonding the flexible tubular housing 20 and/or the optical window 45 to the tip body 40 include epoxies offered by Loctite Corporation under Part No. 4981, by Dymax Corporation under the part number 140-M and by Norland Corporation under part numbers NOA61 and NOA63.

The laser light delivery component 15 includes an optical waveguide 55 disposed within the central lumen 30. The optical waveguide 55 includes an outer protective jacket 56 and a core fiber 57. It is contemplated herein that the laser light delivery component 15 may include one or more other optical waveguides in addition to the optical waveguide 55. A distal end 58 of the core fiber 57 terminates at or within (i.e., is exposed within) the circulation chamber 47. The laser light delivery component 15 further includes a fiber optic coupling 59 (FIG. 1D) for connecting the laser light delivery component 15 to a laser apparatus (not shown in FIGS. 1A through 1D) and a proximal optical waveguide retainer 60 for securing the optical waveguide 55 to a coupling assembly 61 of the energy delivery apparatus 1. A Toughy type bore connector is an example of the proximal optical waveguide retainer 60.

A distal optical waveguide retainer 62 is mounted within the central lumen 30 at the distal end 39 of the flexible tubular housing 20. A thin walled tube is an example of the distal optical waveguide retainer 62. The outer protective jacket 56 of the optical waveguide 55 is stripped back from the distal end 56 of the core fiber 57, exposing the distal end 58 of the core fiber 57. The outer protective jacket 56 is engaged with the distal optical waveguide retainer 62. Engagement of the outer protective jacket 56 with the distal optical waveguide retainer 62 limits forward translation (i.e., towards the optical window 45) of the optical waveguide 55 within the central lumen 30 during flexion of the catheter component 5. Accordingly, the distal end 58 of the core fiber 57 is positioned at a precise distance away from the optical window 45. It will be appreciated that for a given distance between the optical window 45 and the distal end 39 of the flexible tubular housing 20, the distance that the outer protective jacket is stripped back will influence the spot size of the laser light on the optical window 45.

In another embodiment of the catheter component 5 (not shown), a necked-down portion of the central lumen 30 provides the function of the optical waveguide retaining member 60 (i.e., limiting forward displacement of the optical waveguide 55).

In yet another embodiment of the catheter component 5(not shown), the optical waveguide 55 is bonded (e.g., via an adhesive) for precluding translation of the distal end 56 of the optical waveguide 55.

In still another embodiment of the catheter component 5 (not shown), the optical waveguide 55 is adapted for enabling the distal end 58 of the optical waveguide 55 to be controllably translated (i.e. be predictably translated and retained in place) with respect to the flexible tubular housing 20, thereby enabling the distance between the distal end 58 of the core fiber 57 and the optical window 45 to be controllably varied. The ability to controllably translate the distal end 58 of the optical waveguide 55 enables a laser light spot size on the optical window 45 to be controllably varied. For example, the optical waveguide 55 may be mounted in a manner that allows the optical waveguide 55 to be precisely translated within the central lumen 30. In such another embodiment, a means is provided for translating the optical waveguide 55 and for retaining the optical waveguide 55 at a desired position.

Referring to FIG. 1D, the coupling assembly 61 is attached at the proximal end 37 of the flexible tubular housing 20. The coupling assembly 61 includes a cooling medium supply opening 72, a cooling medium return opening 74 and an optical waveguide opening 76. A Toughy bore connector may be implemented at the optical waveguide opening 72 for securing the laser light delivery component 15 to the coupling assembly 61. A cooling medium supply passage 78 extends between the cooling medium supply opening 72 and a first one of the cooling medium lumens 25 for enabling flow of cooling medium from a cooling medium supply apparatus (not shown) through the coupling assembly 61 into the first one of the cooling medium lumens 25. A cooling medium return passage 80 extends between the cooling medium return opening 74 and a second one of the cooling medium lumens 25 for enabling flow of cooling medium from the second one of the cooling medium lumens 25 through the coupling assembly 70 and back to the fluid supply apparatus. The optical waveguide extends through the optical waveguide opening 76.

An embodiment of a cooling medium supply apparatus in accordance with the disclosures made herein may be a circulation type cooling medium supply apparatus or a non-circulation type cooling medium supply apparatus. In a circulation type cooling medium supply apparatus, cooling medium is supplied from a reservoir to the circulation chamber 47 and back to the reservoir. In a non-circulation type cooling medium supply apparatus, cooling medium is supplied from a supply reservoir to the circulation chamber and then to a return reservoir. An example of a non-circulation type cooling medium apparatus system includes a syringe pump, wherein a body of a syringe is a reservoir. Benefits of the use of a syringe pump include ease of metering the cooling medium and reduced potential for contamination of cooling medium being supplied to the circulation chamber. Suitable syringe pumps are commercially available from New Era Pump Systems Corporation and from Harvard Apparatus Corporation.

A first conductor 81 is connected between a first electrical connector 82 and a first feedback variable device (e.g. the tip body 40). A second conductor 83 is connected between a second electrical connector 84 and a second feedback variable device (e.g. the proximal sensing member 49). It is contemplated herein that at least one of the first conductor 81 and the second conductor 82 may extend through a respective passages (not shown) in the coupling assembly 70. It is also contemplated herein that the energy delivery apparatus 1 may include less than two or more than two conductors.

As depicted in FIG. 1A, the catheter component 5 is adapted for enabling a flexure portion 90 of the flexible tubular housing 20 to be controllably deflected between a plurality of positions while an extension portion 92 of the catheter component 5 exhibits minimal deflection resulting from deflection of the flexure portion 90. By providing the extension portion 92 with a higher resistance to deflection than the flexure portion 90, the flexure portion 90 may be deflected (e.g., via a flex wire) without any significant corresponding deflection of the extension portion 92. In one embodiment of the catheter component 5, the flexible tubular housing 20 has essentially uniform flexural properties along its entire length and a sheath (e.g., a piece of shrinkable tubing) is applied over a portion of the flexible tubular housing 20 (i.e., extending from adjacent to the proximal end 37 of the flexible tubular housing 20), thereby defining the extension portion 92 (i.e., the portion with the sheath) and the flexure portion 92. In another embodiment of the catheter component 5, the flexible tubular housing 20 comprises a first length of tube having a relatively high flexural strength (i.e., a first portion of the flexible tubular housing 20) and a second length of tube attached (e.g., thermally bonded) at a distal end of the first length of tube and having a relatively low flexural strength relative to the first length of tube (i.e., a second portion of the flexible tubular housing 20). Accordingly, the first length of tube defines the extension portion 92 and the second length of tube defines the flexure portion 90.

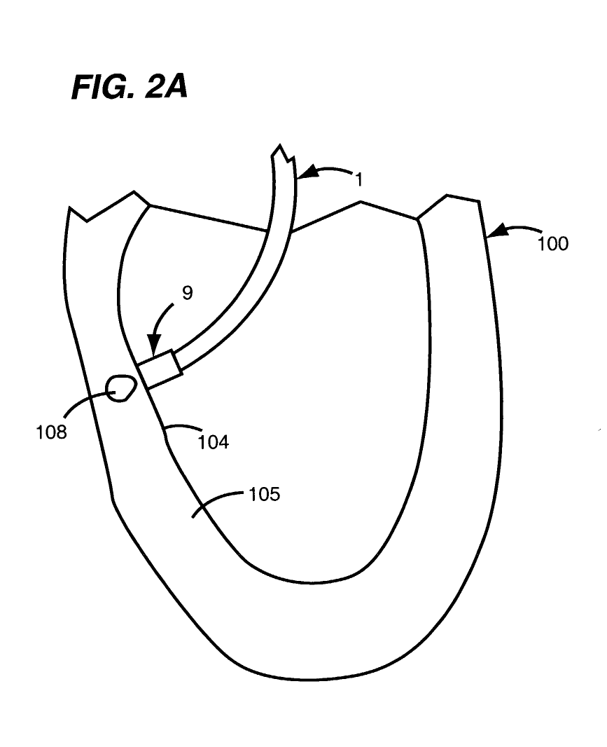

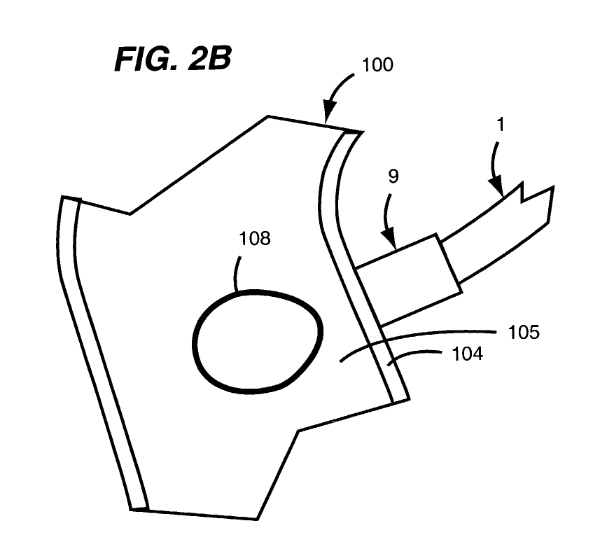

FIGS. 2A and 2B depict an embodiment of a method for utilizing the energy delivery apparatus 1 depicted in FIGS. 1A through 1D. The tip assembly 9 is guided into a heart 100 of a patient and toward an endocardial surface 104 (i.e., a cardiac surface) of the heart 100 directly above diseased tissue. The tip assembly 9 guided into and within the heart 100 via a percutaneous approach under a known guidance approach (e.g., fluoroscopic, MRI, ultrasound, etc). Once at a location to be treated (i.e., above the diseased tissue), the tip assembly 9 is positioned such that the optical window 45 is in direct contact with the endocardial surface 104 of the heart 100. An inside wall 105 of the heart 100 defines the endocardial surface 104.

After the optical window 45 is positioned in direct contact with the endocardial surface 104, laser energy is delivered into the inside wall 105 of the heart 100 after passing from a laser energy source (not shown), through the optical waveguide 55, through cooling medium in the circulation chamber 47 of the tip assembly 9, through the optical window 45 and through the endocardial wall 104. In one embodiment of the laser energy source, the laser energy includes wavelengths between 520 nm and 2100 nm. A preferred wavelength is about 980 nm. A preferred requirement for selecting a wavelength (i.e., the selected wavelength) is that laser light at the selected wavelength is more readily absorbed in water rather in blood. In this manner, tissue will tend to heat at a higher rate than blood when exposed to laser light at the selected wavelength.

It is contemplated herein that before, during and/or after the delivery of laser light, electrical potential may be monitored by measuring the potential between the tip body 40 and the proximal sensing member 49 or any other appropriate electrode attached to the patient (e.g., an Electro-Cardiogram electrode). It is also contemplated herein that an electrical potential may be applied to a heart via the tip body 40. Accordingly, procedures may be conducted for facilitating electro-physiological mapping of a heart. For example, an electrical signal (i.e. an electrical voltage) may be applied via the tip body 40 to one or areas of the heart in accordance with a known pacing protocol, followed by monitoring the tip assembly 9 (via the tip body 40) resulting electrical signals generated by the heart. In this manner, an arrhythmogenic area or arrhythmogenic areas of tissue responsible for generating VT may be identified and mapped (i.e., electro-physiological mapping).

It is contemplated herein that a feedback variable monitoring apparatus is a means adapted for facilitating such electro-physiological mapping. Accordingly, the feedback variable monitoring apparatus is adapted for generating electrical signals for being applied to the heart and monitoring electrical signals generated by the heart. The feedback variable monitoring apparatus is further adapted for mapping electrical signals received from the heart in relation to electrical signals applied to the heart.

During delivery of laser light, a cooling medium is introduced into the energy delivery apparatus 1 such that it flows through the first one of the cooling medium supply lumen 25 (i.e., a cooling medium supply lumen) and into the circulation chamber 47. Accordingly, cooling medium is supplied to the circulation chamber 47 from a cooling medium supply apparatus 9. The cooling medium contacts both the optical window 45 and the optical waveguide 55 within the circulation chamber 47, thus cooling the optical window 45, the optical waveguide 55 and adjacent tissue of the heart 100. Accordingly, both the endocardial surface 104 and core fiber 57 of the optical waveguide 55 are cooled, minimizing the possibility of damaging the tissue comprising the endocardial surface 104 or damaging (e.g., burning or melting) the core fiber 57. The cooling medium flows from the circulation chamber 47 via the second one of the cooling medium lumen 25 (i.e., a cooling medium return lumen). It is contemplated herein that the cooling medium may flow through a plurality of supply lumens and/or a plurality of return lumens.

The temperature and/or flow rate of the cooling medium may be controlled to provide optimum cooling of tissue comprising the endocardial surface 104. It is contemplated herein that the cooling medium may flow in a continuous or in an intermittent manner. Modulating supply temperature and/or flow rate of the cooling medium allows for deposition of maximum photon energy by minimizing or eliminating damage of tissue comprising the endocardial surface 104 of the heart 100, thus leading to development of maximal possible lesions sizes.

Examples of the cooling medium include room temperature and chilled fluids (including gases) such as saline solution, water, air, nitrogen, carbon dioxide and other suitable substances. Suitable substances include fluids (including gases) with a suitable heat capacity and/or that are transmissive to the wavelength of laser light emitted from the optical waveguide 55.

After treatment, the thermal delivery device 1 is removed from the patient and the treated myocardial tissue is allowed to heal. Upon healing, the lesion 108 (FIG. 2B) created in the myocardial tissue using the thermal delivery device 1 prevents (or at least contributes to preventing) initiation or propagation of arrhymogenic signals. The procedure described may be used for creating additional lesions, in addition to the lesion 108 shown. In this manner, ailments such as Ventricular Tachycardia are improved, if not cured.

Use of a cooling medium in accordance with embodiments of the disclosures made herein maintains optical properties of the tissue comprising the inside wall 105 of the heart 100 throughout the deposition of laser light (i.e. photons). By maintaining such optical properties, more photons are able to penetrate the endocardial surface 104 and, hence, to generate temperature increases in deeper layers of tissue comprising the inside wall 105 of the heart 100. The cooling medium carries heat away from the endocardial surface 104 of the heart 100 for preventing adverse temperature rise in the tissue comprising the inside wall 105, thereby limiting adverse changes in the optical properties of the tissue comprising the inside wall 105. The cooling medium also serves to minimize any build up of heat around the optical waveguide 55 and may prevent any thermal damage of the endocardial tissue.

The overall result associated with the use of a cooling medium in accordance with embodiments of the disclosures made herein is the potential for the lesion 108 to be larger and created in a safer manner. Cooling of the tissue comprising the inside wall 105 of the heart 100 limits such tissue from being charred (i.e., carbonized tissue) by overheating at the interface between the optical window 45 and the tissue comprising the inside wall 105 of the heart 100. By limiting the formation of carbonized tissue, forward propagation of photons by direct absorption is not impeded, thus preventing growth of the lesion 108 from being due primarily to conductive heating. By preventing growth of the lesion 108 from being due primarily to conductive heating, the risk for producing blood clots and carbonized tissue with potential for causing life-threatening emboli as they travel through the bloodstream is significantly reduced. Furthermore, there is a reduced potential for the optical waveguide 55 to overheat and cause damage or destruction of components comprising the energy delivery apparatus 1.

During deposition of laser light, one or more feedback variables may be monitored as indicators that adequate tissue damage level has been obtained for forming a desired lesion and for indicating a condition of the endocardial surface 104 and tissue comprising the inside wall 105 of the heart. Examples of feedback variables include surface temperature, electrophysiologic signals, tissue electrical impedance, tissue acoustic impedance, optically monitored colorimetric changes in tissue constituents and mechanical properties such as tissue modulus or compliance. A feedback variable monitoring apparatus in combination with the first conductors, electrical connectors and embodiments of electrode members disclosed in reference to FIGS. 1A through 1D represent means for enabling at least a portion of such feedback variables to be monitored. Such feedback variables may be defined by one or more input signals received by the feedback variable monitoring apparatus.

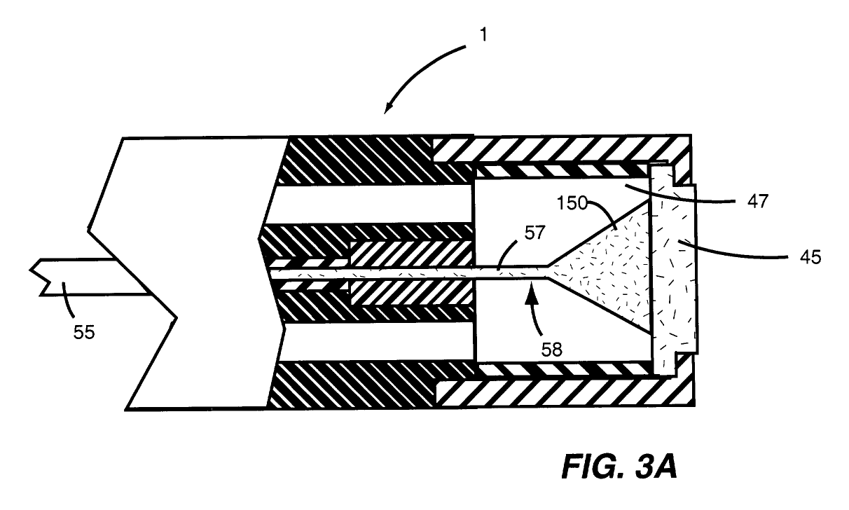

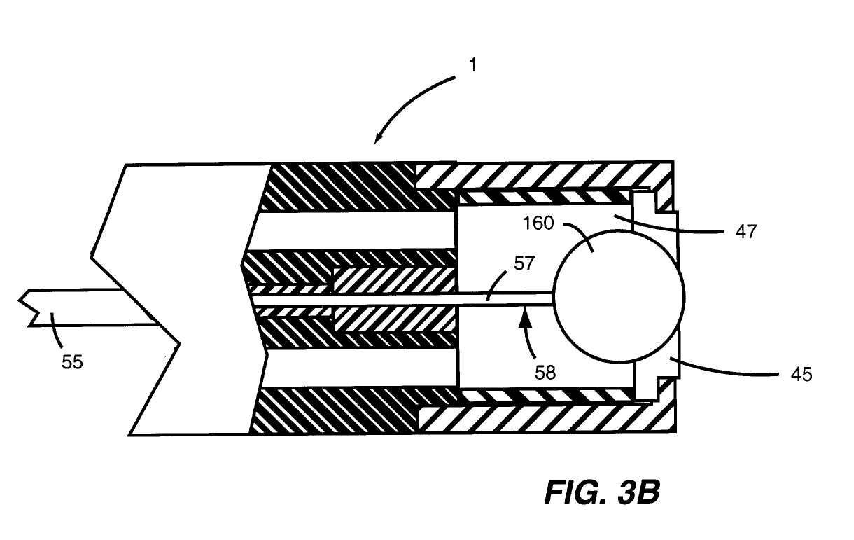

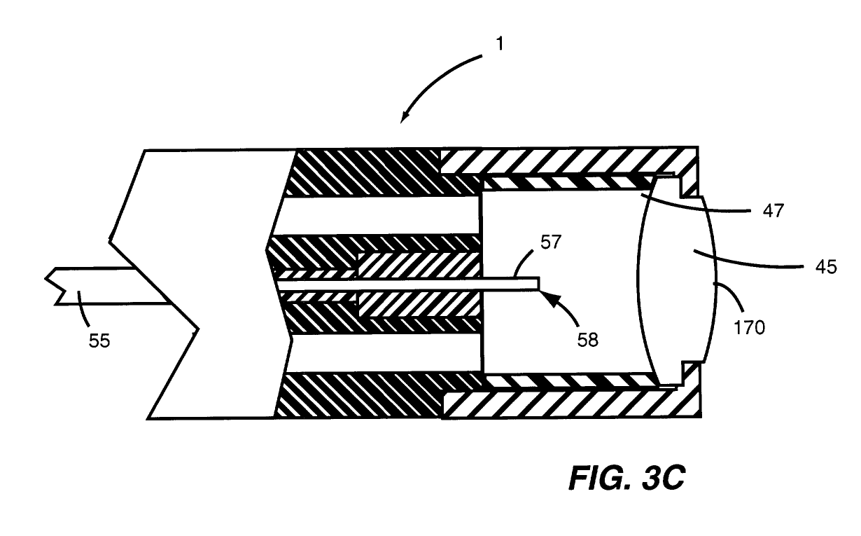

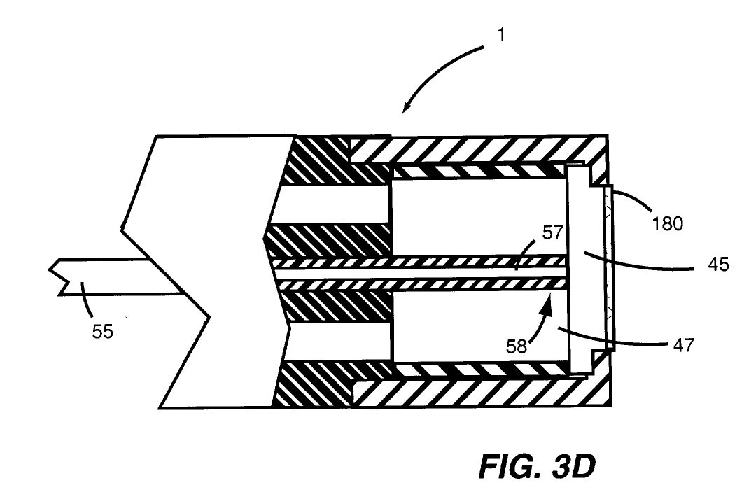

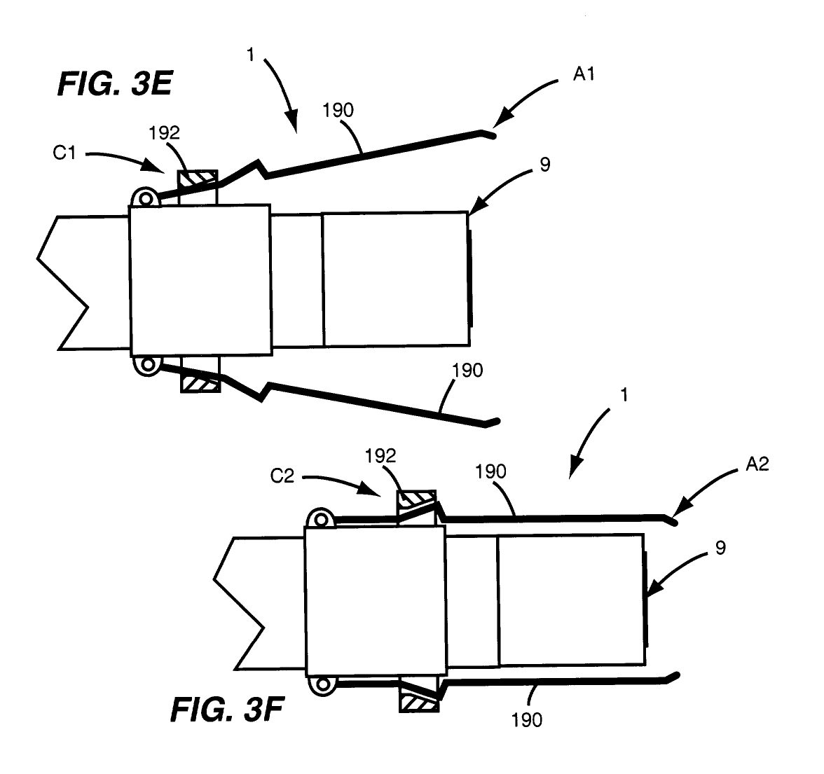

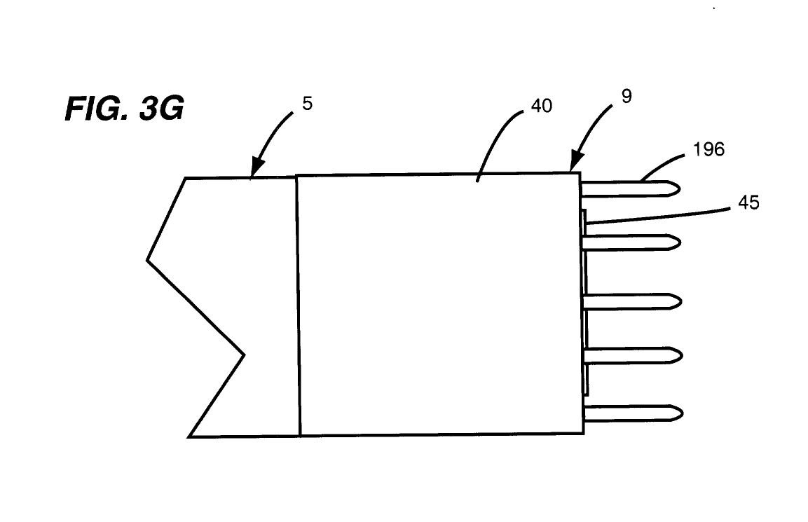

FIG. 3A depicts an alternate embodiment of the energy delivery apparatus 1 wherein the core fiber 57 of the optical waveguide 55 includes a reverse tapered portion 150 at its distal end 58 for providing a larger spot size and reduced power density. The reverse tapered portion 150 may be a discrete component attached at the distal end 58 of the core fiber 57 or may be integrally formed at the distal end 58 of the core fiber 57. The reverse tapered portion 150 is preferably engaged with the optical window 45. The reverse tapered portion 150 is adapted for allowing lower power use due to a decrease in light attenuation from the cooling medium circulating within the circulation chamber 47.

FIG. 3B depicts an alternate embodiment of the energy delivery apparatus 1 wherein the core fiber 57 of the optical waveguide 55 includes a ball-ended lens 160 at the distal end 58 of the core fiber 57. The ball-ended lens 160 is adapted for allowing laser light to be focused in a prescribed manner within tissue comprising an inside wall of a heart. The ball-ended lens 160 may be a discrete component attached at the distal end 58 of the core fiber 57 or may be integrally formed at the distal end 58 of the core fiber 57. The ball-ended lens 160 is preferably engaged with the optical window 45.

FIG. 3C depicts an alternate embodiment of the energy delivery apparatus 1 wherein the optical window 45 includes a contoured surface 170, thereby shaping the optical window 45 to define a lens. By shaping at least one surface the optical window 45 to define a lens, laser light is focused within tissue comprising an inside wall of a heart in a manner that contributes to protecting the endocardial surface of the heart from thermal damage. Accordingly, deeper lesions may be safely and effectively produced. It is contemplated herein that the optical window 45 may be shaped to provide any one of a number of lens configurations. For example, the optical window 45 may be shaped to define a convex-shaped lens, a concave-shaped lens, a concave-convex shaped lens or other suitable shape lens.

FIG. 3D depicts an alternate embodiment of the energy delivery apparatus 1 wherein the optical window 45 includes at least one surface 180 adapted to diffuse laser light. The core fiber 57 of the optical waveguide 55 is preferably engaged with the optical window 45 within the circulation chamber 47. Various types of means for modifying a surface of a transmissive body to diffuse incident light are known. By diffusing the laser light, a lower power density is effectively produced while preventing any significant light attenuation within cooling medium circulating within the circulation chamber 47.

FIGS. 3E and 3F depicts an alternate embodiment of the energy delivery apparatus 1 including a plurality of arms 190. The arms 190 are examples of tip retaining members. The arms 190 may be essentially straight or substantially curved. The arms 190 are adapted for grasping tissue at a treatment site at a cardiac surface, thereby aiding in maintaining contact between the optical window 45 and a treatment site. It is contemplated herein that a single arm may be provided for aiding in maintaining contact between the optical window 45 and the treatment site. It is also contemplated herein that the plurality of arms 190 may be self-retracting or manually retracting. For example, a collar 192 may be movable between a disengaged position C1 (FIG. 3E) and an engaged position C2 (FIG. 3F). Moving the collar 192 from the disengaged position P1 to the engaged position P2 facilitates moving the arms 190 from respective retracted positions A1 to respective engaged positions A2. In addition to aiding in maintaining contact between the optical window 45 and a treatment site, the arms 190 may also serve to increase heat transfer by increasing contact area, thus aiding in extracting heat from the treatment site.

FIG. 3G depicts an alternate embodiment of the energy delivery apparatus 1 including a plurality of protruding members 196. The protruding members 196 are examples of tip retaining members. The protruding members 196 extend from the tip assembly 9 (e.g., attached to the tip body 40 or to the optical window 45). The protruding members 196 reduce the potential for unintentional movement of the tip assembly 9 when the tip assembly 9 is engaged with a treatment site of a heart. It is contemplated herein that the protruding members 196 may be fixed attached or retractably attached to the tip assembly 9. In addition to reduce the potential for unintentional movement of the tip assembly 9 at the treatment site, the protruding members 196 may also serve to increase heat transfer by increasing contact area, thus aiding in extracting heat from the treatment site.

### EXAMPLE 1

### Cooled Tip Laser Catheter System Construction

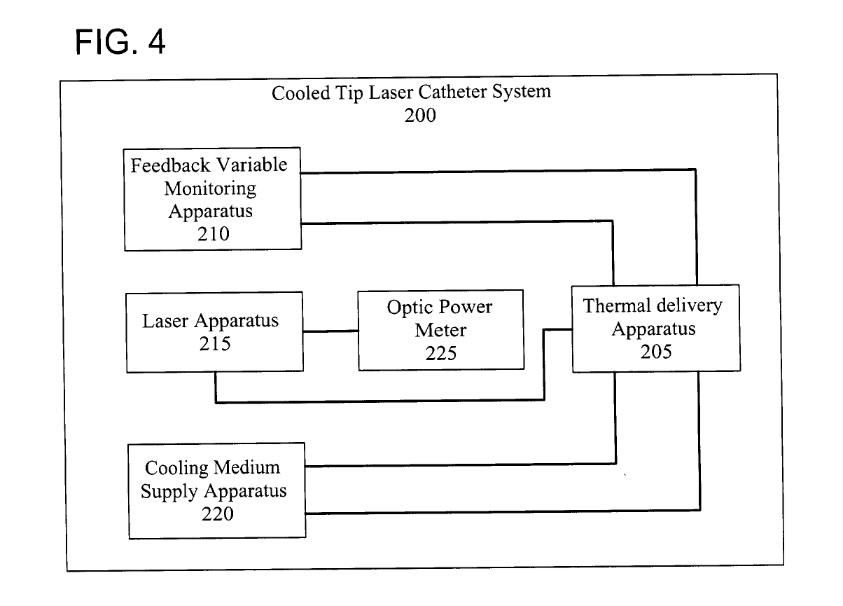

A Cooled Tip Laser Catheter (CTLC) system in accordance with an embodiment of the disclosures made herein was designed and built for facilitating creation of therapeutic lesions in a heart necessary to treat ventricular tachycardia. FIG. 4 depicts an embodiment of a Cooled Tip Laser Catheter (CTLC) system 200 in accordance with the disclosures made herein. The CTLC system 1 includes an energy delivery apparatus 205 (e.g., the energy delivery apparatus 1 depicted in FIGS. 1A through 1D), a feedback variable monitoring apparatus 210, a laser apparatus 215, a cooling medium supply apparatus 220 and an optic power meter 225. The feedback variable monitoring apparatus 210 and the cooling medium supply apparatus are attached directly to the energy delivery apparatus 205. The energy delivery apparatus 205 and the optic power meter 225 are connected in parallel with the laser apparatus 215. In other embodiment (not shown), light emitted from a distal end of the energy delivery apparatus 205 measured directly by the optic power meter 225 (e.g., by directing laser light from the energy delivery apparatus 205 on a sensor of the optic power meter 225.

The feedback variable monitoring apparatus 210 is adapted for enabling one or more feedback variables to be monitored and/or logged. The laser apparatus 215 is adapted for enabling laser light to be delivery to the energy delivery apparatus 205. The cooling medium supply apparatus 220 is adapted for facilitating delivery/return of cooling medium to/from the energy delivery apparatus 205. The optic power meter 225 is adapted for enabling a power level of laser light from the laser apparatus to be monitored and/or logged.

Various specific aspects the design and construction of the CTLC system 200 are presented below.

Energy Delivery Apparatus

A length of multi-lumen polyurethane tubing extruded by Putnam Plastics Corporation (Dayville, Conn.) served as a flexible tubular housing of a catheter component of the energy delivery apparatus. The polyurethane tubing incorporated a barium-doped formulation to render the tubing housing radio-opaque under fluoroscopy. The outer diameter of the tubing was nominally 2.5 mm and included a single central lumen (about 0.85 mm) surrounded by 6 smaller lumens (about 0.5 mm).

A sapphire window measuring 2.5 mm in diameter and 0.5 mm in thickness was procured from Edmund Industrial Optics (Barrington, N.J.). The sapphire window served as an optical window of the energy delivery apparatus. The optical window was bonded within a 5.75 mm long stainless steel tube (i.e., a tip body), thus forming a tip assembly of the energy delivery apparatus. The sapphire window exhibits a high thermal conductivity of 33 W/m degrees K and a relatively large surface area over which heat removal from the endocardial surface of the heart may occur.

A low-OH hard clad multimode optic fiber was procured from 3M Company Specialty Fibers Division (Westhaven, Conn.) and was mounted within the central lumen (i.e., an optic fiber lumen) of the tubing housing. The optic fiber served as an optical waveguide of the energy delivery apparatus. The overall diameter of the clad multimode optic fiber was 730 μm and the diameter of the optic fiber itself was 400 μm. The numerical aperture of the optic fiber was 0.39, which allowed efficient light coupling and excellent transmission performance during tight bends of the optic fiber.

The stainless steel tube was attached to a distal end of the polyurethane tubing. A circulation chamber is defined within the stainless steel tube between the optical window and the end of the polyurethane tubing. The tip assembly was attached to the polyurethane tubing with the optical waveguide and the optical window separated by a distance resulting in a laser spot size of between about 1.25 mm and 1.5 mm on the sapphire window. Graph 1 (below) depicts calculated results showing 89% transmission (including losses due to interfacial reflections) at a separation distance required to produce a 1.5 mm diameter spot on the face of the sapphire window. The selection of a spot size of about 1.5 mm was due in part to modeling discussed herein below.

[image]

A first pair of the smaller lumens (i.e., cooling medium supply lumens) was used for carrying cooling medium from a cooling medium supply apparatus to the circulation chamber. A second pair of the smaller lumens (i.e., cooling medium return lumens) was used for carrying cooling medium from the circulation chamber back to the cooling medium supply apparatus. A remaining one of the smaller lumens was used to carry a small thermocouple for monitoring temperature of the optical window and the cooling medium within the circulation chamber.

One of the smaller lumens was used to carry a 0.015″ stainless steel wire (i.e., a flex wire) for facilitating flexing of the tubing housing. A tri-female Luer Y-connector was attached to the proximal end of the polyurethane tubing for providing a means of connecting to the cooling medium supply lumens, to the supply medium lumens and to the optic fiber lumen.

A custom machined plastic handle was provided at a proximal end of the tubing housing. A first end of the flex wire was attached to the stainless steel tube and a second end of the flex wire was attached to a slideable actuator of the handle. The length of the polyurethane tubing, excluding a length of 5 cm at the distal end of the polyurethane tubing, was covered with a thin layer (0.001″) of polyester heat shrink (i.e., a sheath) procured from Advanced Polymers (Salem, N.H.). The sheath provided increased stiffness and a junction where the polyurethane tubing would bend when flexed. Movement of the slideable actuator on the handle provides a mechanism for flexing a distal end of the tubing housing and, thus, the tip assembly, by an angle of approximately 120 degrees relative to a longitudinal axis of the sheathed portion of the polyurethane tubing.

Cooling Medium Supply Apparatus

A commercially-available automotive fuel pump was used with a control circuit for controlling flow of cooling medium within the circulation chamber. The pump is relatively small in size, runs on a standard 12V DC power supply, and provides sufficient flow rate for the cooling requirements of the energy delivery apparatus. The pump was removable submersed within a 5-Liter reservoir of saline solution-based cooling medium, thus providing cooling medium to the pump. Flow controls were used to redirect any flow not sent to the catheter back into the reservoir, thus controlling the flow rate through the circulation chamber. A flow rate of between 15-30 ml/min was achievable through the circulation chamber. This pump mounting configuration allowed easy removal of fluids and cleaning when needed.

Laser Apparatus

A commercially-available, low-powered diode laser was used for supplying laser energy. Specifically, a laser light output portion of a diode laser device was connected to a core fiber of the optical waveguide. The laser operated at 980 nm and was powered via normal 110 VAC line voltage. An optic power meter was attached to the laser for monitoring power output of the laser. The laser was specified as being adapted for delivering up to 5W at 980 nm through a 200 μm optic fiber. The 980 nm wavelength provides significant penetration depth and heat generation in myocardial tissue and, therefore, is well suited for creating deep therapeutic lesions.

Thermal Modeling

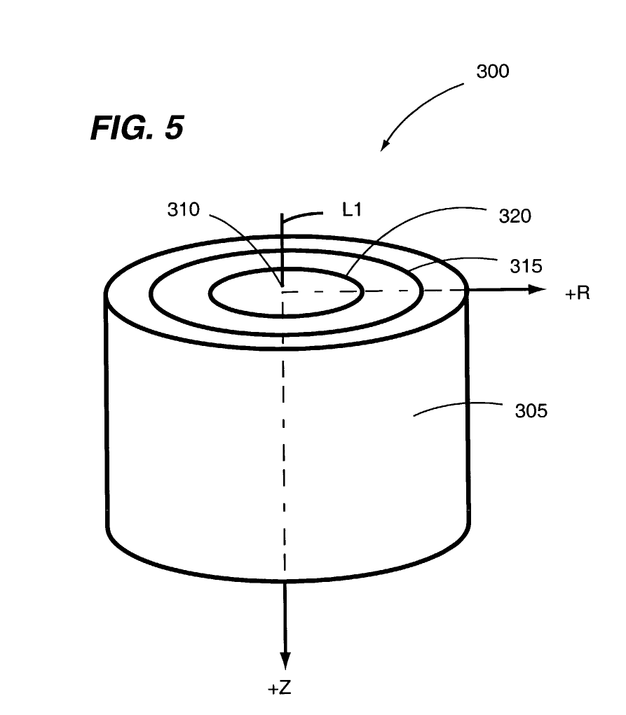

Thermal modeling was performed via a mathematical model formulated for approximating optical-thermal response of the energy delivery apparatus of Example 1. A geometric representation 300 of the energy delivery apparatus of Example 1 (i.e., the subject energy delivery apparatus) is depicted in FIG. 5. Canine myocardium is represented as a cylinder 305 with an origin 310 of a coordinate system located on a face of the cylinder at a longitudinal axis L1 of the cylinder 305. The optical window is represented as a first circle 315 encompassed by the canine myocardium (i.e., a circle defined by the cylinder 305). The incident laser beam emitted in a Z-direction with respect to the myocardium (i.e., along the longitudinal axis L1 of the cylinder 305) is represented as a second circle 320 encompassed by the first circle 315. The first circle has a 2.5 mm diameter and the second circle has a 1.5 mm diameter (i.e. representing a 1.5 mm spot size on the optical window).

Due to the symmetry of the model, cylindrical coordinates were used so that angular dependences were eliminated. Light distribution was calculated using diffusion approximation of a conventional light transport equation. The resulting heat transfer within the myocardium was modeled with a two-dimensional heat conduction equation in cylindrical coordinates. Due to the short time scales associated with delivering thermal energy via the energy delivery apparatus, thermal effects from metabolic heat generation and blood perfusion were assumed to be negligible. Published optical properties for myocardial tissue at 980 nm were not available. Accordingly, optical properties for canine myocardium at 810 nm and 1064 nm were used to determine fluence rate profiles within this model.

After validation of the model in terms of fluence distributions and temperature profiles, simulations were run to investigate the effects of spot size on fluence and temperature distributions. The effects of temperature profiles due to changes in contact resistance of an optical window made of sapphire were also investigated.

Graph 2 (below) depicts resulting normalized fluence rate profiles for spot sizes of 1.0, 1.5, and 2.0 mm for a constant incident irradiance.

[image]

Graphs 3 and 4 (below) depict the rate of heat generation as a function of depth along the z-axis for several spot sizes for both non-cooled (Graph 3) and cooled (Graph 4) conditions.

[image]

[image]

The resulting rate of heat generation illustrates the effect of heat generation being inversely proportional to the square of the spot radius and directly proportional to the fluence rate. Accordingly, surface cooling is more effective at lowering temperature when the spot size of the incident beam is 1.5 mm compared to either 1.0 or 2.0 mm. The heat transfer coefficient of the sapphire (hₛₐₚₚ) is set to 0 in the model (i.e. when no heat transfer between the sapphire and myocardium), temperature predicted by the model is approximately 4 times higher at the surface than when a high value for hₛₐₚₚ (i.e. when good thermal transfer between the sapphire and myocardium) is used. It should be appreciated that while Graphs 3 and 4 are useful for demonstrating trends, it should be noted that the temperature values in this model are predicted to be relatively high due to an over-estimation of absorption coefficient, which could not be specifically identified from publicly available literature.

After heuristic correction of the optical absorption parameter, 2-D model simulations were performed for both the cooled and uncooled case with a 1.5 mm spot size. It should be noted that in the cooled case, maximum temperatures are reached at locations below the incident surface (i.e. z>0). This is consistent with the morphology of lesions observed in both in vitro and in vivo studies (discussed below).

Through the use of the subject energy delivery apparatus and thermal modeling, a number of objects of energy delivery apparatuses in accordance with the disclosures made herein were proven. One object proven through the use of the subject energy delivery apparatus was that power density of laser irradiation on treated tissue may be decreased sufficiently and surface tissue temperature may be sufficiently reduced, thus limiting char formation of tissue at an endocardial surface of the heart. Another object proven through the use of the subject energy delivery apparatus was that a relatively small diameter fiber optic maintains flexibility of a catheter component. Still another object proven through the use of the subject energy delivery apparatus was that laser energy is effectively transmitted through a cooling medium within a circulation chamber with minimal loss. Yet another object proven through the use of the subject energy delivery apparatus was that sufficient thermal heat transfer between the irradiated myocardial tissue and an tip assembly is achievable via heat transfer through the tip assembly (i.e., via an annular surface of a tip body and a face of an optical window).

### EXAMPLE 2

### In Vitro Studies

The Cooled Tip Laser Catheter (CTLC) system of Example 1 (i.e., the subject system) was utilized for performing in vitro studies on blood perfused canine myocardium. The intent of such studies was to establish optimum cooling rates and laser dosimetry for producing maximal lesion sizes. To this end, in vitro studies were designed to define suitable cooling rates and laser doses for which a therapeutic lesion size could be achieved with minimal or no charring at the endocardial surface.

In preliminary experiments, it was determined that the tissue temperature of an organ is the most important variable in maintaining physiologic conditions during in vitro experiments. Accordingly, an environmental chamber made of plastic sheet material and surrounded by a heated water jacket was used to maintain heart tissue at a normal body temperature of 37 degrees C.

Following these preliminary experiments, a number of in vitro studies were performed via in vitro tissue samples consisting of whole canine and beef hearts. The results of these in vitro studies are presented below.

Dosimetry Studies

A goal of a myocardial ablation apparatus such as the subject system is achieving lesions sizes that provide therapeutic effects. As mentioned previously, production of char at an endocardial surface is a deterrent to penetration of laser photons to the deeper layers, thus inhibiting the formation of lesions with larger coagulation depths. An effective way to avoid char initiation as well as water vaporization is to maintain lower temperatures at the endocardial-catheter juncture.

Evaluations were performed for determining threshold exposure times for char formation for a range of parameters of the energy delivery apparatus. This threshold exposure information was useful in developing an understanding of upper threshold exposure times where undesirable effects might result. Temperature measurements for monitoring the interfacial temperature and gross inspection at the tissue-fiber interface was used to determine these threshold exposure times.

Cooling fluid was water fixed at room temperature (25° C.). Laser power values of 3, 4 and 5 Watts and coolant flow rates of 0, 15, and 30 ml/min were used. Results from these studies were used to guide parameters for in vitro dosing studies.

In vitro dose response data were generated for laser powers and exposure times determined in preliminary studies to demonstrate safe and effective use of the cooled tip approach. Lesions were made by operating the energy delivery apparatus at 3 and 4 watts of laser power for exposure times of 60, 90, 120, and 180 seconds. In these studies, the cooling fluid was maintained at room temperature (25° C.) with flow rate through the catheter of 15 ml/min and laser spot size was fixed at 1.5 mm. A total of 40 lesions were made in these studies (n=5 lesions/dose). For each combination, the lesion width and depth were measured by gross examinations after the samples were bisected along their length. The optical window of the subject system was inspected closely for coagulated blood that may have been trapped between optical window and the endocardium. Graph 5 and Graph 6 (below) show the results from these studies relative to 3 Watt and 4 Watt laser power, respectively.

[image]

[image]

### EXAMPLE 3

### In Vivo Studies

To evaluate an ability to make therapeutic lesions in a safe and efficient manner with the Cooled Tip Laser Catheter (CTLC) system of Example 1 (i.e., the subject system), studies were conducted using the subject system in vivo on a canine model from an endocardial approach. Both acute and chronic animal studies were used to assess the performance of the subject system under in vivo conditions where perfusion in the ventricle chamber and heart tissue could affect resulting temperature distributions. In addition, it was important to assess the performance of the catheter component of the subject system on a beating heart to ensure the catheter remained in position and that cooling effects were maintained during delivery of laser energy.

The canine model was chosen due to its similarity in terms of anatomy, size, hemodynamics, and optical properties with that of the human heart. Both acute and chronic (2 wk, 4 wk, and 8 wk) studies were performed. Acute studies were designed to demonstrate the feasibility of creating lesions with a desirable morphology and location for treatment of VT and to begin identifying the optimal dosing parameters for use with the subject system in vivo. Subsequent to the acute studies, chronic experiments were performed. The chromic experiments were designed to identify any proarrhythmia potential or other early rhythm complications induced by creation of lesions using the subject system.

Acute Studies

Four (4) mongrel dogs weighing 15-30 kg were used in the acute studies. The dogs were anesthetized with butorphanol (0.2-0.4 mg/kg IM) and propofol (4-6 mg/kg IV), intubated, and ventilated with 0.25-2% isoflurane. A femoral artery catheter and skin electrodes were placed for continuous monitoring of blood pressure and cardiac rhythm respectively. For simplicity, the left or right carotid artery was surgically exposed, and endocardial access was obtained by placing a 9 French catheter sheath under fluoroscopic guidance into the left ventricle. The catheter introducer sheath was then used for introduction of the catheter component of the subject system into the heart. After placement of the introducer sheath a left lateral thoracotomy or median stemotomy (at the surgeon's discretion) was performed and the heart suspended in a pericardial cradle thereby exposing the left ventricular surface. The exposed epicardium facilitated location of the exact treatment site.

Prior to making lesions with the subject system, a visible aiming beam (670 nm diode laser) was activated which passed through the tissue and allowed us to mark the position of the probe with a small epicardial suture. This method facilitated identification of lesions after treatment and eliminated overlap of serial lesions. All lesions in these in vivo studies were created using the fiber-coupled 980 nm diode laser previously described above in reference to the construction of the subject system. A total of 26 lesions were placed in the 4 animals undergoing acute experiments. Laser powers of 3 and 4 Watts with exposure times of 60, 90, 120, and 180 seconds were used in these studies.

All lesions were allowed to mature for 1 hour after which time the animals were sacrificed by injection of sodium pentobarbital. Hearts were removed and rinsed in cold saline. Lesions were bisected and measured grossly followed by removing a block of tissue containing the lesion and submitting it for histopathological analysis.

Lesions were well tolerated by animals in all cases. Transient episodes of tachycardia were noted upon contact of the catheter component with the endocardium. These episodes were generally short lived and not present during delivery of laser energy. Table 1 (below) summarizes results of lesion dimensions found in these acute studies. Lesion volumes presented in Table 1 were calculated assuming ellipsoidal geometry.

| | | Mean | Mean | Mean |
|---|---|---|---|---|
| | Exposure | Lesion | Lesion | Lesion |
| Laser | Time | Depth | Width | Volume* |
| Power | (Seconds) | (mm) | (mm) | (mm³) |
| 3W | 60 | 5.8 | 6.2 | 116 |
| | 90 | 6.3 | 6.9 | 172 |
| | 120 | 5.9 | 7.0 | 151 |
| | 180 | 6.2 | 9.1 | 268 |
| 4W | 90 | 7.2 | 6.6 | 164 |
| | 120 | 7.3 | 7.8 | 248 |
| | 180 | 6.4 | 8.5 | 242 |

Lesion Parameters for Acute Animal Studies

Subendocardial lesions created with the subject system in vivo were located on average 0.95 mm below the endocardial surface. In most cases, lesions were roughly spherical in shape and at the time of sectioning were characterized by a well-defined border of hyperemia. There was virtually no sign of thermal damage to the endocardial surface. Yet, when endocardial tissues were bisected, large myocardial lesions were present. The lesions were well-defined and absent of char, carbonization or other evidence of overheating. For one representative sample of the lesions, this lesion extended to a depth of 7.4 mm with a maximum width of 8.5 mm.

Histological Analysis for Acute Studies

Histology results confirmed well-circumscribed focus of thermal injury with a sharp boundary of contraction band necrosis between the lesion and normal myocardium and minimal endocardial damage.

Effect of Tissue Cooling

In a single acute animal, the effects of the cooled-tip approach on performance of the subject system was demonstrated. Immediately after sacrifice of the single acute animal, two lesions were produced from the epicardial surface of the exposed heart in areas of the left ventricle away from lesions made in vivo. Results from this demonstration were dramatic. At 4 Watts of laser power, a lesion produced without cooling resulted in burning of the tissue and tip of the catheter component after only 24 seconds of energy delivery. This was in stark contrast to relatively mild surface damage achievable with cooling resulting after 180 seconds of energy delivery.

Chronic Studies (5 Animals):

A total of 5 animals (1 control, 4 treated) were used in chronic experiments. The anesthetic protocol for the animals in the chronic studies was identical to that used in acute studies. In addition, a bupivicaine intercostal block was used to minimize any post-operative discomfort. Use of the subject system in acute studies was essentially duplicated in the chronic studies, except that aseptic techniques were used and animals were recovered upon completion of the lesion creation procedure.

A primary goal for the chronic studies was to determine the potential for early rhythm disturbances resulting from creation of lesions using the subject system. To this end, ambulatory electrocardiographic (Holter) monitoring was performed on all animals following treatment. A single control animal in which no lesions were made was used to determine baseline rhythm disturbances resulting from marking the epicardium with sutures for lesion location. In chronic animals, approximately 3 to 4 lesions were created in each animal with the subject system using a set laser power of 3W and exposure times of 60 to 90 seconds.

After placement of lesions, the catheter component was removed, the carotid artery ligated, and the thoracotomy closed routinely. Following the surgery animals were placed in Holter monitors and electocardiographic data was acquired for a 24-hour period. Chronic animals were subsequently sacrificed at 2 (n=1), 4 (n=1), and 8 (n=3: 1 control) weeks at which time their hearts were removed, fixed in 10% buffered formalin, and submitted for histopathological analysis.

Histological Analysis for Chronic Studies

Endocardium of the hearts of the chronic animals were presented as normal. The lesion from a 3W/60 second exposure produced in a two-week chronic animal was relatively large (approximately 9 mm in diameter), located in the midmyocardium, and surrounded by fibroblasts and mononuclear inflammatory cells. At 4 weeks, a similarly created lesion consisted of a discrete area of dense connective tissue. This appearance is consistent with a chronic healing response for thermal induced myocardial lesions. In all cases, there was no significant chronic histological appearance on the endocardial surface associated with the midmyocardial lesions produced with the subject system.

Assessment of Early Rhythm Disturbances

To more completely evaluate the acute effects of the use of the CTLC system of Example 1 (i.e., the subject system) on cardiac rhythm, 24-hour ambulatory electrocardiograms via Holter monitor were performed on all 5 (i.e., including the 1 control) of the chronic animals. Ambulatory electrocardiograms were recorded 24 hours prior to induction of lesions and then again in the initial 24 hours after the procedure. Particular attention was paid to the number and morphology of premature ventricular complexes (PVC's) recorded. The recording device was a standard six electrode (three lead) ambulatory electrocardiogram and full disclosure analysis was performed using a Delmar™ analysis system.

The number of the PVC's noted on baseline recordings varied from 0 to 254 in 24 hours. In all cases, these PVC's were isolated and of a single morphology. This number of PVC's is within the normal range of what is seen in clinically normal dogs. The number of PVC's noted in the initial 24 hours following induction of lesions ranged from 2 to 773 in 24 hours. Again, the PVC's noted were typically isolated and of single morphology. In one animal, PVC's occasionally occurred in pairs and in two instances in a short run of ventricular tachycardia (maximum heart rate <160 beats per minute).

Based on the findings of the 24-hour ambulatory electrocardiograms it appears that in the immediate postoperative period the laser-induced lesions have minimal effects on cardiac rhythm. It must be considered that some of the postoperative rhythm disturbances detected may be associated with the anesthesia and median sternotomy performed in the experiment. This is supported by the fact that the control animal also had a slight increase in the number of PVC's noted on the post-procedural ambulatory electrocardiogram. These results of the in vivo studies provide insight into the acute effects of procedures with the subject system on rhythm in animals with initially normal myocardium.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. To avoid unnecessary detail, the description omits certain information known to those skilled in the art. For example, certain dimensions of elements of an infusion device, certain orientations of elements, specific selection of materials for various elements and the like may be implemented based on an engineering preference and/or a specific application requirement. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. An energy delivery apparatus, comprising:
a flexible tubular housing including a plurality of lumens therein extending between a proximal end and a distal end of the flexible tubular housing;
a tip assembly including a tip body attached at a first end thereof to the distal end of the flexible tubular housing and an optical window mounted at a second end of the tip body, wherein a circulation chamber is defined within the tip body between the distal end of the flexible tubular housing and the optical window; and
an optical waveguide mounted within a first of said lumens, wherein a distal end of the optical waveguide is exposed within the circulation chamber.

2. The apparatus of claim 1 wherein the optical waveguide extends approximately along a longitudinal axis of the flexible tubular housing.

3. The apparatus of claim 1 wherein the flexible tubular housing is adapted for being radio-opaque under fluoroscopy.

4. The apparatus of claim 1 wherein the flexible tubular housing is formed from a material having a formulation capable of rendering the flexible tubular housing radio-opaque under fluoroscopy.

5. The apparatus of claim 4 wherein the formulation includes a radio-opaque doping material.

6. The apparatus of claim 1 wherein:
the flexible tubular housing includes an optical waveguide retaining member mounted in the first one of said lumens adjacent to the distal end of the flexible tubular housing; and
the optical waveguide includes an outer protective jacket having a face thereof engaged with the optical waveguide retaining member.

7. The apparatus of claim 1, further comprising:
means for limiting translation of the optical waveguide with respect to a longitudinal axis of the flexible tubular housing, thereby positioning the distal end of the optical waveguide at an essentially fixed position with respect to the optical window.

8. The apparatus of claim 1 wherein a second one of lumens has a truncated semi-circular cross-sectional shape.

9. The apparatus of claim 8 wherein the second one of said lumens is exposed within the circulation chamber.

10. The apparatus of claim 1 wherein:
a second one and a third one of said lumens have a truncated semi-circular cross-sectional shape; and
the first one of said lumens is positioned between the second one and the third one of said lumens.

11. The apparatus of claim 1 wherein:
the first one of said lumens is a centrally located lumen with respect to a plurality of peripherally located ones of said lumens; and
at least a portion of said plurality of peripherally located ones of said lumens are exposed within the circulation chamber.

12. The apparatus of claim 1 wherein:
a first portion of the flexible tubular housing defines a flexure portion of the flexible tubular housing;
a second portion of the flexible tubular defines an extension portion of the flexible tubular housing; and
the flexure portion is adapted for being controllably deflected between a plurality of positions while the extension portion exhibits minimal deflection resulting from deflection of the flexure portion.

13. The apparatus of claim 12 wherein:
the flexure portion is made from a material having a first flexural strength; and
the extension portion is made from a material having a second flexural strength, different than the first flexural strength.

14. The apparatus of claim 13 wherein an end of the first portion of the flexible tubular housing is connected to an end of the second portion of the flexible tubular housing.

15. The apparatus of claim 1 further comprising:
a sheath mounted on an exterior surface of an extension portion of the flexible tubular housing, thereby providing the extension portion of the flexible tubular housing with a flexural strength different than a flexural strength of a flexure portion of the flexible tubular housing.

16. The apparatus of claim 1 wherein the tip body includes a shoulder portion that engages a mating shoulder portion of the optical window.

17. The apparatus of claim 16 wherein the tip assembly includes an optical window support member within the circulation chamber between the optical window and the distal end of the flexible tubular housing.

18. The apparatus of claim 1 wherein a face of the optical window and a face of the tip body are essentially flush.

19. The apparatus of claim 1, further comprising:
a plurality of protruding members, wherein each one of said protruding members is attached to at least one of the tip body and the optical window.

20. The apparatus of claim 19 wherein a longitudinal axis of each one of said protruding members extends generally parallel with a longitudinal axis of the tip body.

21. The apparatus of claim 1 wherein:
the optic waveguide includes a core fiber; and
the core fiber extends into the circulation chamber.

22. The apparatus of claim 1 wherein the optic waveguide is adapted for being controllably translated within the first one of said lumens.

23. The apparatus of claim 1, further comprising;
a lens attached at the distal end of the optic waveguide.

24. The apparatus of claim 1 wherein the optical window includes a surface adapted for diffusing light.

25. The apparatus of claim 24 wherein:
the optic waveguide includes a core fiber; and
the core fiber is in contact with the optical window.

26. The apparatus of claim 1, further comprising:
a coupling assembly attached at the proximal end of the flexible tubular housing, wherein the coupling assembly is adapted for enabling a cooling medium to be supplied to the circulation chamber via a second one of said lumens and returned from the circulation chamber via a third one of said lumens.

27. The apparatus of claim 1 wherein the coupling assembly includes:
a cooling medium supply passage aligned with a second one of said lumens; and
a cooling medium return passage aligned with a third one of said lumens.

28. The apparatus of claim 1, further comprising:
a flex wire attached to the tip assembly and extending between distal end of the flexible tubular housing and the proximal end of the flexible tubular housing through one of said lumens.

29. The apparatus of claim 28, wherein:
the tip body is electrically conductive; and
the flex wire is electrically connected to the tip body.

30. The apparatus of claim 1, further comprising:
means for maintaining contact between the optical window and an interior wall of a heart.

31. The apparatus of claim 30, further comprising:
a tip retaining member attached to the tip assembly and adapted for maintaining contact between the optical window and an interior wall of a heart.

32. The apparatus of claim 31 wherein the tip retaining member is movably attached to the tip body and is capable of being moved between a retracted position and an engaged position.

33. An energy delivery apparatus, comprising:
a flexible tubular housing including a plurality of lumens therein extending between a proximal end and a distal end of the flexible tubular housing, wherein a first one of said lumens has a circular cross-sectional shape, a second one of said lumens has a truncated semi-circular cross-sectional shape and a longitudinal axis of the first one of said lumens extends approximately along a longitudinal axis of the flexible tubular housing;
a tip assembly including a tip body attached at a first end thereof to the distal end of the flexible tubular housing and an optical window mounted at a second end of the tip body, wherein a circulation chamber is defined within the tip body between the distal end of the flexible tubular housing and the optical window;
an optical waveguide mounted within the first of said lumens, wherein the optic waveguide includes a core fiber having a distal end disposed within the circulation chamber; and
means for limiting translation of the optical waveguide with respect to a longitudinal axis of the flexible tubular housing, thereby positioning the distal end of the core fiber at an essentially fixed position with respect to the optical window.

34. An energy delivery apparatus, comprising:
a tip assembly including a tip body having opposed ends and light transmissive means adapted for transmitting laser light mounted at a first one of said opposed ends of the tip body, wherein a circulation chamber is defined within the tip body between said opposed ends
positioning means for enabling the tip assembly to be guided within a heart and engaged with a cardiac surface of the heart, wherein a distal end of said positioning means is attached to a second one of said opposed ends of the tip body;
heat removal means for enabling heat to be removed from said light transmissive means; and means for directing laser light through said light transmissive means.

35. A cooled tip laser catheter system, comprising:
an energy delivery apparatus including:
a flexible tubular housing including a plurality of lumens therein extending between a proximal end and a distal end of the flexible tubular housing;
a tip assembly including a tip body attached at a first end thereof to the distal end of the flexible tubular housing and an optical window mounted at a second end of the tip body, wherein a circulation chamber is defined within the tip body between the distal end of the flexible tubular housing and the optical window;
an optical waveguide mounted within a first of said lumens, wherein a distal end of the optical waveguide is exposed within the circulation chamber;
a laser apparatus attached to the energy delivery apparatus in a manner enabling laser light to be supplied to and transmitted by the optical waveguide; and
a cooling medium supply apparatus attached to the energy delivery apparatus in a manner enabling cooling medium to be circulated through the circulation chamber.

36. The system of claim 35 wherein the laser apparatus includes a diode laser device.

37. The system of claim 36 wherein the diode laser device is adapted for emitting light having a wavelength between about 520 nm and about 2100 nm.

38. The system of claim 36 wherein the diode laser device is adapted for emitting light having a wavelength of about 980 nm.

39. The system of claim 36 wherein a laser light output portion of the diode laser device is connected to a core fiber of the optical waveguide.

40. The system of claim 35 wherein the cooling medium supply apparatus includes a pump attached to a second one of said lumens.

41. The system of claim 35 wherein the cooling medium supply apparatus includes a flow control device adapted for limiting flow of said cooling medium to the circulation chamber.

42. The system of claim 35 wherein the cooling medium supply apparatus is adapted for controlling at least one of a temperature and a flow rate of said cooling medium.

43. The system of claim 35 wherein the cooling medium supply apparatus is adapted for enabling at least one of continuous flow and intermittent flow of said cooling medium.

44. The system of claim 35 wherein the cooling medium supply apparatus is a circulation type cooling medium supply apparatus.

45. The system of claim 35 wherein the cooling medium supply apparatus is a non-circulation type cooling medium supply apparatus.

46. The system of claim 45 wherein the non-circulation type cooling medium supply apparatus includes a syringe pump.

47. The system of claim 35, further comprising:
a feedback variable monitoring apparatus attached to the tip assembly.

48. The system of claim 47 wherein:
the feedback variable monitoring apparatus is adapted for at least one of generating an electrical signal for being applied to a heart and monitoring an electrical signal generated by the heart; and
the tip body is adapted for applying the electrical signal generated by the feedback variable monitoring apparatus to the heart and for enabling the electrical signal generated by the heart to be conducted to the feedback variable monitoring apparatus.

49. The system of claim 48 wherein the feedback variable monitoring apparatus is further adapted for mapping an electrical signal received from the heart in relation to an electrical signal applied to the heart.

50. The system of claim 35 wherein the feedback variable monitoring apparatus is adapted for monitoring at least one of an input signal related to surface temperature, an input signal relating to an electro-physiologic signal, an input signal relating to tissue electrical impedance, an input signal relating to tissue acoustic impedance, an input signal relating to optically monitored calorimetric changes in tissue constituents and an input signal relating to a tissue mechanical property.

51. The system of claim 35 wherein a first signal input of the feedback variable monitoring apparatus is attached to the tip body.

52. The system of claim 51 wherein the tip body is electrically conductive.

53. The system of claim 51, further comprising:
a sensing member attached to the flexible tubular housing between the tip body and the proximal end of the flexible tubular member, wherein a second signal input of the feedback variable monitoring apparatus is attached to the sensing member.

54. A cooled tip laser catheter system, comprising:
an energy delivery apparatus including:
a flexible tubular housing including a plurality of lumens therein extending between a proximal end and a distal end of the flexible tubular housing;
a tip assembly including a tip body attached at a first end thereof to the distal end of the flexible tubular housing and an optical window mounted at a second end of the tip body, wherein a circulation chamber is defined within the tip body between the distal end of the flexible tubular housing and the optical window; and
an optical waveguide positioned within a first of said lumens, wherein a distal end of the optical waveguide is exposed within the circulation chamber;
a laser device attached to the energy delivery apparatus in a manner enabling laser light to be supplied to and transmitted by the optical waveguide;
a syringe pump attached to a lumen of the energy delivery apparatus for enabling a cooling medium to be supplied to the circulation chamber; and
a feedback variable monitoring apparatus attached to the tip assembly, wherein the feedback variable monitoring apparatus is adapted for generating an electrical signal for being applied to a heart, for monitoring an electrical signal generated by the heart and for mapping an electrical signal received from the heart in relation to an electrical signal applied to the heart;
wherein the tip body is adapted for applying the electrical signal generated by the feedback variable monitoring apparatus to the heart and for enabling the electrical signal generated by the heart to be conducted to the feedback variable monitoring apparatus.

55. A method for treating a cardiac condition, comprising:
engaging an optical window of an energy delivery apparatus against a cardiac surface of a heart;
directing laser light through the optical window while the optical window is engaged against the cardiac surface, wherein the laser light is transmitted through an optical waveguide of the energy delivery apparatus, and
supplying cooling medium to a circulation chamber of the energy delivery apparatus while the optical window is engaged against the cardiac surface, wherein the optical window at least partially defines the circulation chamber and a distal end of the optical waveguide is exposed within the circulation chamber.

56. The method of claim 55 wherein:
engaging the optical window includes guiding a tip assembly of the energy delivery apparatus within the heart from a percutaneous approach under fluoroscopy; and
the tip assembly includes the optical window.

57. The method of claim 55 wherein engaging the optical window includes positioning the optical window above an arrhythmogenic focus.

58. The method of claim 55 wherein directing laser light includes transmitting laser light from a laser device through an optical waveguide.

59. The method of claim 55 wherein directing laser light includes maintaining a distal end of the optical waveguide at a fixed distance from the optical window.

60. The method of claim 55, further comprising:
at least temporarily inhibiting said directing laser light in response to a temperature monitored at a tip assembly of the energy delivery apparatus exceeding a prescribed level.

61. The method of claim 55 wherein directing laser light includes maintaining a constant laser output power.

62. The method of claim 55 wherein directing said laser light includes modulating laser output power dependent upon feedback variable information.

63. The method of claim 55 wherein supplying said cooling medium includes adjusting a cooling medium flow rate dependent upon at least one feedback variable being monitored via the tip member.

64. The method of claim 55 wherein supplying said cooling medium includes intermittently supplying said cooling medium.

65. The method of claim 55 wherein supplying said cooling medium includes adjusting a cooling medium flow rate dependent upon a temperature of at least one of the optical window and a tip member of the energy delivery apparatus.

66. The method of claim 55 wherein supplying said cooling medium includes supplying said cooling medium to the circulation chamber after cooling said cooling medium.

67. The method of claim 66 wherein cooling said cooling medium includes reducing the temperature of said cooling medium to within a prescribed cooling medium temperature range.

68. The method of claim 55 wherein supplying said cooling medium includes circulating at least one of saline solution, water, air, nitrogen and carbon dioxide.

69. The method of claim 55, further comprising;
monitoring feedback variable information while directing said laser light.

70. The method of claim 69 wherein monitoring said feedback variable information includes monitoring said feedback variable information via a tip assembly of the energy delivery apparatus.

71. The method of claim 69 wherein monitoring said feedback variable information includes monitoring at least one of an input signal related to surface temperature, an input signal relating to an electro-physiologic signal, an input signal relating to tissue electrical impedance, an input signal relating to tissue acoustic impedance, an input signal relating to optically monitored colorimetric changes in tissue constituents and an input signal relating to a tissue mechanical property.

72. The method of claim 71 wherein directing said laser light includes modulating laser output power dependent upon said at least a portion of said feedback variable information.

73. The method of claim 55, further comprising:
applying an apparatus-generated electrical signal to the heart after engaging the optical window;
monitoring an electrical signal generated by the heart in response to the applying the apparatus-generated electrical signal.

74. The method of claim 73 wherein the tip body is adapted for applying the apparatus-generated electrical signal to the heart and for enabling the electrical signal generated by the heart to be conducted to the feedback variable monitoring apparatus.

75. The method of claim 73, further comprising mapping the electrical signal received from the heart in relation to the electrical signal applied to the heart.

76. A method for treating a cardiac condition, comprising:
engaging an optical window of an energy delivery apparatus against a cardiac surface of a heart;
applying an apparatus-generated electrical signal to the heart after engaging the optical window;
monitoring an electrical signal generated by the heart in response to the applying the apparatus-generated electrical signal;
mapping the electrical signal received from the heart in relation to the electrical signal applied to the heart.
directing laser light through the optical window while the optical window is engaged against the cardiac surface, wherein the laser light is transmitted through an optical waveguide of the energy delivery apparatus, and
supplying cooling medium to a circulation chamber of the energy delivery apparatus while the optical window is engaged against the cardiac surface, wherein the optical window at least partially defines the circulation chamber and a distal end of the optic component is exposed within the circulation chamber.

77. A method for treating a cardiac condition, comprising:
performing a tip positioning process for engaging an optical window of an energy delivery apparatus against a cardiac surface of a heart;
performing a laser light transmission process for imparting energy into tissue of the heart below the cardiac surface, wherein the laser light transmission process includes transmitting said laser light through an optical waveguide of the energy delivery apparatus, and
performing a cooling process removing heat from the optical window and from a distal end of the optical waveguide.
